(19) United States
(12) Patent Application Publication
(10) Pub. No.: US 2014/0278523 A1
(43) Pub. Date: Sep. 18, 2014

(54) DYNAMICALLY ASSOCIATING AND DISASSOCIATING PATIENTS AND MEDICAL DEVICES

(71) Applicant: CERNER INNOVATION, INC., Lenexa, KS (US)

(72) Inventor: JAY CHRISTOPHER VAGLIO, KANSAS CITY, KS (US)

(73) Assignee: CERNER INNOVATION, INC., LENEXA, KS (US)

(21) Appl. No.: 13/796,483

(22) Filed: Mar. 12, 2013

(51) Int. Cl. G06Q 50/24; A61B 5/00

(52) U.S. Cl. CPC G06Q 50/24; A61B 5/0002
USPC 705/3; 340/539.12

(57) ABSTRACT

Systems, methods, computer storage media, and user interfaces are provided for dynamically associating and disassociating patients and medical devices. A signal communicated via a Bluetooth personal area network is received when a patient is in proximity to a medical device. In various embodiments, the signal originates with a patient wristband or the medical device. The medical device is automatically associated to the patient. When the signal is no longer being received, the medical device may be automatically disassociated from the patient. Data associated with the medical device may be communicated to an EMR associated with the patient. A mobile device associated with a clinician may be communicated with via the Bluetooth personal area network.

### CROSS REFERENCE TO RELATED APPLICATIONS

This application is related to commonly assigned U.S. patent application entitled “Associating Patients and Medical Devices with a Mobile Device via Bluetooth” (Attorney Docket CRNI.182544), filed concurrently herewith on the same date.

### BACKGROUND

Typically, medical devices that are used to treat or care for a patient are not adequately or timely linked to that patient in the patient's record, such as an electronic medical record (EMR). In many instances, this lack of linkage or association may lead to many inaccuracies and inconsistencies in treating the patient. The lack of association between a patient and medical devices used to treat the patient may necessitate multiple queries in order to locate certain information related to the patient's treatment. For instance, even if a particular patient's record is queried and found, data from the medical devices used in conjunction with the patient's treatment may not be included in the record, but may require separate and multiple queries. In some cases, the data from the medical devices may be very difficult, or even impossible to locate. Further, time related to the associations (e.g., patient to device, clinician to patient, and the like) is not currently trackable without manual effort. For example, a facility or insurance provider may desire to track time a medical device was actually associated with a patient. In another example, the facility or insurance provider may desire to track a clinician's interaction with the medical device and patient. Such tracking of time may be extremely useful for analytics but is largely dependent on documentation provided by the clinician.

The current workflow of associating devices to patients, for the purposes of logging data to an electronic medical record (EMR) associated with the patient, is a cumbersome process. The process requires manual scanning of patients and devices and is often confusing and not aligned with the natural workflow of a clinician. Often, priority is given to patient care and the clinician must associate devices after the fact (i.e., retro-association). The manual process required for retro-association, particularly when attempting to track time of associations for analytic purposes, introduces many opportunities for human error.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. The present invention is defined by the claims.

Embodiments of the present invention provide systems, methods, computer storage media, and user interfaces for dynamically associating and disassociating patients and medical devices. A signal via a Bluetooth personal area network (PAN) is received when a patient is in proximity to a medical device. In various embodiments, the signal originates with a patient wristband or the medical device. The medical device is automatically associated to the patient. When the Bluetooth signal is no longer being received, the medical device is automatically disassociated from the patient. In embodiments, data associated with the medical device is communicated to an EMR associated with the patient. In embodiments, a mobile device associated with a clinician is communicated with via the Bluetooth PAN.

### BRIEF DESCRIPTION OF THE DRAWINGS

Embodiments are described in detail below with reference to the attached drawing figures, wherein:

FIG. 1 is a block diagram of an exemplary computing environment suitable for use in implementing embodiments of the present invention;

FIG. 2 is an exemplary system architecture suitable for use in implementing embodiments of the present invention;

FIG. 3 is an illustrative screen display showing a patient summary display area, in accordance with an embodiment of the present invention;

FIG. 4 is an illustrative screen display showing a connection display area, in accordance with an embodiment of the present invention;

FIG. 5 is an illustrative screen display showing an association confirmation display area, in accordance with an embodiment of the present invention;

FIG. 6 is an illustrative screen display showing device display area, in accordance with an embodiment of the present invention;

FIG. 7 is an illustrative screen display showing a disconnected display area, in accordance with an embodiment of the present invention;

FIG. 8 is an illustrative screen display showing a disassociation confirmation display area, in accordance with an embodiment of the present invention;

FIG. 9 is an illustrative screen display showing a shift log display area, in accordance with an embodiment of the present invention;

FIG. 10 is an illustrative flow diagram of a method for dynamically associating and disassociating patients and medical devices, in accordance with an embodiment of the present invention;

FIG. 11 is an illustrative flow diagram of a method for dynamically associating and disassociating patients and medical devices, in accordance with an embodiment of the present invention; and

FIG. 12 is an illustrative flow diagram of a method for associating patients and medical devices with a mobile device via Bluetooth, in accordance with an embodiment of the present invention.

### DETAILED DESCRIPTION

The subject matter of the present invention is described with specificity herein to meet statutory requirements. However, the description itself is not intended to limit the scope of this patent. Rather, the inventors have contemplated that the claimed subject matter might also be embodied in other ways, to include different steps or combinations of steps similar to the ones described in this document, in conjunction with other present or future technologies. Moreover, although the terms “step” and/or “block” may be used herein to connote different elements of methods employed, the terms should not be interpreted as implying any particular order among or between various steps herein disclosed unless and except when the order of individual steps is explicitly described.

Embodiments of the present invention provide systems, methods, computer storage media, and user interfaces for, among other things, dynamically associating and disassociating patients and medical devices. A signal via a Bluetooth PAN is received when a patient is in proximity to a medical device. In various embodiments, the signal originates with a patient wristband or the medical device. The medical device is automatically associated to the patient. When the Bluetooth signal is no longer being received, the medical device is automatically disassociated from the patient. In embodiments, a mobile device associated with a clinician is communicated with via the Bluetooth PAN.

In various embodiments of the present invention, data from a medical device may be communicated to a patient's EMR while the patient is associated with the medical device. As utilized herein, the acronym “EMR” is not meant to be limiting, and may broadly refer to any or all aspects of the patient's medical record rendered in a digital format. Generally, the EMR is supported by systems configured to co-ordinate the storage and retrieval of individual records with the aid of computing devices. As such, a variety of types of healthcare-related information may be stored and accessed in this way. By way of example, the EMR may store one or more of the following types of information: patient demographic; medical history (e.g., examination and progress reports of health and illnesses); medicine and allergy lists/immunization status; laboratory test results, radiology images (e.g., X-rays, CTs, MRIs, etc.); evidence-based recommendations for specific medical conditions; a record of appointments and physician's notes; billing records; and data received from an associated medical device. Accordingly, systems that employ EMRs reduce medical errors, increase physician efficiency, and reduce costs, as well as promote standardization of healthcare.

Accordingly, one embodiment of the present invention is directed to one or more computer storage media storing computer-useable instructions that, when used by one or more computing devices, cause the one or more computing devices to perform a method. The method comprises: receiving a signal, at a medical device, the signal originating from a patient wristband associated with a patient in proximity to the medical device and communicated via a Bluetooth PAN; recognizing the patient wristband associated with the signal; identifying a patient associated with the patient wristband; and associating the patient to the medical device.

In another embodiment, the present invention is directed to a system for automatically associating a patient to a medical device. The system comprises: a patient wristband associated with a patient that communicates to one or medical devices via a Bluetooth PAN when the patient wristband is within range of the one or more medical devices; the one or more medical devices configured to communicate via the Bluetooth PAN with a mobile device associated with a clinician; the mobile device configured to receive an association request to associate the one or more medical devices to the patient; and an electronic medical record associated with the patient configured to receive device information communicated by the one or more medical devices and clinician information from the mobile device associated with the clinician.

In yet another embodiment, the present invention is directed to a method for associating a medical device to a patient. The method comprises: receiving, at a patient wristband, a signal originating from a medical device, the signal being communicated via a Bluetooth PAN; automatically associating the medical device to the patient; and automatically disassociating the medical device from the patient when the patient wristband no longer receives the signal.

Having briefly described embodiments of the present invention, an exemplary operating environment suitable for use in implementing embodiments of the present invention is described below. Referring to the drawings in general, and initially to FIG. 1 in particular, an exemplary computing system environment, for instance, a medical information computing system environment, with which embodiments of the present invention may be implemented is illustrated and designated generally as reference numeral 100. It will be understood and appreciated by those of ordinary skill in the art that the illustrated medical information computing system environment 100 is merely an example of one suitable computing environment and is not intended to suggest any limitation as to the scope of use or functionality of the invention. Neither should the medical information computing system environment 100 be interpreted as having any dependency or requirement relating to any single component or combination of components illustrated therein.

The present invention may be operational with numerous other general purpose or special purpose computing system environments or configurations. Examples of well-known computing systems, environments, and/or configurations that may be suitable for use with the present invention include, by way of example only, personal computers, server computers, hand-held or laptop devices, multiprocessor systems, microprocessor-based systems, set top boxes, programmable consumer electronics, network PCs, minicomputers, mainframe computers, distributed computing environments that include any of the above-mentioned systems or devices, and the like.

The present invention may be described in the general context of computer-executable instructions, such as program modules, being executed by a computer. Generally, program modules include, but are not limited to, routines, programs, objects, components, and data structures that perform particular tasks or implement particular abstract data types. The present invention may also be practiced in distributed computing environments where tasks are performed by remote processing devices that are linked through a communications network. In a distributed computing environment, program modules may be located in association with local and/or remote computer storage media including, by way of example only, memory storage devices.

With continued reference to FIG. 1, the exemplary medical information computing system environment 100 includes a general purpose computing device in the form of a control server 102. Components of the control server 102 may include, without limitation, a processing unit, internal system memory, and a suitable system bus for coupling various system components, including database cluster 104, with the control server 102. The system bus may be any of several types of bus structures, including a memory bus or memory controller, a peripheral bus, and a local bus, using any of a variety of bus architectures. By way of example, and not limitation, such architectures include Industry Standard Architecture (ISA) bus, Micro Channel Architecture (MCA) bus, Enhanced ISA (EISA) bus, Video Electronic Standards Association (VESA) local bus, and Peripheral Component Interconnect (PCI) bus, also known as Mezzanine bus.

The control server 102 typically includes therein, or has access to, a variety of computer-readable media, for instance, database cluster 104. Computer-readable media can be any available media that may be accessed by server 102, and includes volatile and nonvolatile media, as well as removable and non-removable media. By way of example, and not limitation, computer-readable media may include computer storage media and communication media; computer storage media excluding signals per se. Computer storage media may include, without limitation, volatile and nonvolatile media, as well as removable and non-removable media implemented in any method or technology for storage of information, such as computer-readable instructions, data structures, program modules, or other data. In this regard, computer storage media may include, but is not limited to, RAM, ROM, EEPROM, flash memory or other memory technology, CD-ROM, digital versatile disks (DVDs) or other optical disk storage, magnetic cassettes, magnetic tape, magnetic disk storage, or other magnetic storage device, or any other medium which can be used to store the desired information and which may be accessed by the control server 102. Communication media typically embodies computer-readable instructions, data structures, program modules, or other data in a modulated data signal, such as a carrier wave or other transport mechanism, and may include any information delivery media. As used herein, the term “modulated data signal” refers to a signal that has one or more of its attributes set or changed in such a manner as to encode information in the signal. By way of example, and not limitation, communication media includes wired media such as a wired network or direct-wired connection, and wireless media such as acoustic, RF, infrared, and other wireless media. Combinations of any of the above also may be included within the scope of computer-readable media.

The computer storage media discussed above and illustrated in FIG. 1, including database cluster 104, provide storage of computer-readable instructions, data structures, program modules, and other data for the control server 102. The control server 102 may operate in a computer network 106 using logical connections to one or more remote computers 108. Remote computers 108 may be located at a variety of locations in a medical or research environment, for example, but not limited to, clinical laboratories (e.g., molecular diagnostic laboratories), hospitals and other inpatient settings, veterinary environments, ambulatory settings, medical billing and financial offices, hospital administration settings, home health care environments, and clinicians' offices. Clinicians may include, but are not limited to, a treating physician or physicians, specialists such as surgeons, radiologists, cardiologists, and oncologists, emergency medical technicians, physicians' assistants, nurse practitioners, nurses, nurses' aides, pharmacists, dieticians, microbiologists, laboratory experts, laboratory technologists, genetic counselors, researchers, veterinarians, students, and the like. The remote computers 108 may also be physically located in non-traditional medical care environments so that the entire health care community may be capable of integration on the network. The remote computers 108 may be personal computers, servers, routers, network PCs, peer devices, other common network nodes, or the like, and may include some or all of the elements described above in relation to the control server 102. The devices can be personal digital assistants or other like devices.

Exemplary computer networks 106 may include, without limitation, local area networks (LANs) and/or wide area networks (WANs). Such networking environments are commonplace in offices, enterprise-wide computer networks, intranets, and the Internet. When utilized in a WAN networking environment, the control server 102 may include a modem or other means for establishing communications via the WAN, such as the Internet. In a networked environment, program modules or portions thereof may be stored in association with the control server 102, the database cluster 104, or any of the remote computers 108. For example, and not by way of limitation, various application programs may reside on the memory associated with any one or more of the remote computers 108. It will be appreciated by those of ordinary skill in the art that the network connections shown are exemplary and other means of establishing a communications link between the computers (e.g., control server 102 and remote computers 108) may be utilized.

In operation, a clinician may enter commands and information into the control server 102 or convey the commands and information to the control server 102 via one or more of the remote computers 108 through input devices, such as a keyboard, a pointing device (commonly referred to as a mouse), a trackball, or a touch pad. Other input devices may include, without limitation, microphones, satellite dishes, scanners, or the like. Commands and information may also be sent directly from a remote healthcare device to the control server 102. In addition to a monitor, the control server 102 and/or remote computers 108 may include other peripheral output devices, such as speakers and a printer.

Although many other internal components of the control server 102 and the remote computers 108 are not shown, those of ordinary skill in the art will appreciate that such components and their interconnection are well known. Accordingly, additional details concerning the internal construction of the control server 102 and the remote computers 108 are not further disclosed herein.

As previously mentioned, embodiments of the present invention provide systems, methods, computer storage media, and user interfaces for dynamically associating and disassociating patients and medical devices. A signal via a Bluetooth PAN is received when a patient is in proximity to a medical device. In various embodiments, the signal originates with a patient wristband or the medical device. The medical device is automatically associated to the patient. When the signal is no longer being received, the medical device is automatically disassociated from the patient. In embodiments, data associated with the medical device is communicated to an EMR associated with the patient. In embodiments, a mobile device associated with a clinician is communicated with via the Bluetooth PAN.

As also previously mentioned, embodiments of the present invention provide systems, methods, computer storage media, and user interfaces for associating patients and medical devices with a mobile device via a Bluetooth PAN. A signal associated with a patient communicated via a Bluetooth PAN is received. The patient associated with the signal is recognized. An association request to associate the patient to a medical device is initiated and communicated to a mobile device associated with a clinician when the mobile device is in range. Once the clinician has provided an indication that the association request is approved, the patient is associated to the medical device. When a signal is received that the patient is out of range or disconnected from the device, a disassociation request may be initiated. The disassociation request may be communicated to the mobile device and once the clinician has provided an indication that the disassociation request is approved, the patient is disassociated from the medical device. In embodiments, the mobile device is selected based on an assignment and/or proximity of the clinician. In embodiments, while the patient is associated with the medical device, data associated with the medical device is automatically stored in an EMR associated with the patient. In embodiments, data is automatically logged in a shift log associated with the clinician while the mobile device is communicating with the medical device or the signal associated with the patient via the Bluetooth PAN.

Referring now to FIG. 2, a block diagram is provided illustrating an exemplary computing system 200 suitable for use in implementing embodiments of the present invention. Generally, the computing system 200 allows for communication via a network 202 between medical devices 210, 212, 214, mobile devices 220, 222, a patient wristband 230, an EMR 240, and an association engine 250. The functionality provided by association engine 250 may be distributed across one or more of the medical devices 210, 212, 214, the mobile device 220, 222, the patient wristband 230, or may be provided by a standalone computing device, such as server 102 as illustrated in FIG. 1. Medical devices may include any medical device that could be used to treat a patient, or any devices or mechanisms that may be used by a patient during a hospital stay or doctor's office visit, for example. These medical devices may include, for exemplary purposes only, a patient's bed, monitors (e.g., fetal monitors), pumps (e.g., infusion pump), cardiac ventilators, sequential compression devices, electronic security devices, and the like.

Each of medical devices 210, 212, 214, mobile devices 220, 222, the patient wristband 230, the EMR 240, and the association engine 250 may communicate via the network 202 utilizing the same or different communication protocols. The network 202 may include, without limitation, one or more local area networks (LANs), one or more wide area networks (WANs), and/or one or more PANs. For example, the medical devices 210, 212, 214, mobile devices 220, 222, and the patient wristband 230 may communicate via a Bluetooth PAN. The EMR 240 and the association engine 250, on the other hand, may communicate with medical devices 210, 212, 214, mobile devices 220, 222, or the patient wristband 230 utilizing different communication protocols, such as a combination of wireless and wired methodologies.

It should be understood that any number or type of medical devices 210, 212, 214, mobile devices 220, 222, and/or association engines 250 may be employed in the computing system 200 within the scope of embodiments of the present invention. Each may comprise a single device/interface or multiple devices/interfaces cooperating in a distributed environment. For instance, the association engine 250 may comprise multiple devices and/or modules arranged in a distributed environment that collectively provide the functionality of the association engine 250 described herein. Additionally, other components or modules not shown also may be included within the computing system 200.

In some embodiments, one or more of the illustrated components/modules may be implemented as stand-alone applications. In other embodiments, one or more of the illustrated components/modules may be implemented via medical devices 210, 212, 214, mobile devices 220, 222, the patient wristband 230, association engine 250, or as an Internet-based service. It will be understood by those of ordinary skill in the art that the components/modules illustrated in FIG. 2 are exemplary in nature and in number and should not be construed as limiting. Any number of components/modules may be employed to achieve the desired functionality within the scope of embodiments hereof. Further, components/modules may be located on and/or shared by any number of association engine 250 and/or medical devices 210, 212, 214, mobile devices 220, 222, the patient wristband 230. By way of example only, the association engine 250 might be provided as a single computing device (as shown), a cluster of computing devices, or a computing device remote from one or more of the remaining components.

It should be understood that this and other arrangements described herein are set forth only as examples. Other arrangements and elements (e.g., machines, interfaces, functions, orders, and groupings of functions, etc.) can be used in addition to or instead of those shown, and some elements may be omitted altogether. Further, many of the elements described herein are functional entities that may be implemented as discrete or distributed components or in conjunction with other components, and in any suitable combination and location. Various functions described herein as being performed by one or more entities may be carried out by hardware, firmware, and/or software. For instance, various functions may be carried out by a processor executing instructions stored in memory.

Referring still to FIG. 2, the association engine 250 is configured to, among other things, dynamically associate and disassociate patients and medical devices. The association engine 250 is additionally configured to, among other things, associate patients and medical devices via Bluetooth. As illustrated, in various embodiments, the association engine 250 includes a detection component 252, an identifier component 254, an association request component 256, an approval component 258, an association component 260, a time component 262, a shift log component 264, a disassociation request component 266, and a disassociation component 268.

The patient wristband 230 associated with a patient communicates to one or more medical devices 210, 212, 214 via a Bluetooth PAN when the patient wristband is within range of the one or more medical devices. For example, the patient may enter a room in a particular facility or unit that includes one or more medical devices 210, 212, 214. Once the patient wristband 230 and the one or more medical devices 210, 212, 214 are within range of one another, the patient wristband and/or the medical devices may communicate a signal. When the signal is received, the devices “pair” and a Bluetooth PAN is established. In one embodiment, detection component 252 detects the signal communicated from the patient wristband to the medical device or from the medical device to the patient wristband. In one embodiment, detection component 252 receives an indication the medical device has detected a signal associated with the patient. The signal indicates the patient is within range of the medical device and may be communicated from the patient wristband 230 to the medical device 210, 212, 214 via the Bluetooth PAN. Communication of the signal initiates an association request to the mobile device, such as by the association request component 256 described below. The association request may include a patient identifier associated with the patient.

The one or more medical devices are configured to communicate with a mobile device 220, 222 via the Bluetooth PAN. The mobile device may be associated with a clinician that is also within range of the patient wristband 230 and the one or more medical devices 210, 212, 214. The mobile device 220, 222 is configured to receive an associate request to associate the one or more medical devices to the patient. In other words, although the patient wristband and the medical devices are paired and the Bluetooth PAN has been established, the patient and a particular medical device is not actually associated until approved by the clinician. The selection of the clinician may be based on a role or location of the clinician. The location of the clinician may be determined by the proximity of the mobile device associated with the clinician to the patient wristband and medical devices.

An EMR 240 associated with the patient is configured to receive device information communicated by the one or more medical devices and/or clinician information communicated by the mobile device associated with the clinician. The medical devices and/or the mobile device may communicate with the EMR via any of the communication protocols described herein. In addition, the EMR may further be configured to receive patient information communicated by the patient wristband.

The identifier component 254 of the association engine 250 is configured to identify, in one embodiment, the patient associated with the signal. The signal may contain a patient identifier that helps the identifier component 254 identify the patient. The patient identifier may be encrypted or otherwise void of patient identifiable information such that only the identifier component 254 can identify the patient. In other words, the patient identifier included with the signal may only have meaning to the identifier component 254 and may not be useable by any other component of association engine 250.

The association request component 256 of the association engine 250 is configured to initiate an association request for the patient to the medical device. The association request is communicated to a mobile device associated with a clinician. The association request identifies the patient and any medical devices that have successfully paired.

The approval component 258 of the association engine 250 is configured to receive an indication of an approval from the clinician for the association request. The approval may be communicated by the mobile device to the approval component 258 via the Bluetooth PAN or any other available communication protocol. If one or more medical devices are included in the request, the approval component 258 receives an indication of an approval for each of the one or more medical devices. In other words, a clinician may desire to associate the patient to less than all of the medical devices included in the request. The clinician is able to approve the association for the patient to the medical device on an individual, per device basis (effectively, allowing the clinician the ability to provide an indication of a disapproval for at least a portion of the association request).

The association component 260 of the association engine 250 is configured to associate the patient to the medical device. Once the indication of approval is received by the approval component 258, the associate component 260 associates the patient to the medical device. This association enables data from the medical device to be communicated to an EMR associated with the patient as described above.

In one embodiment, the time component 262 of the association engine 250 is configured to track time in a time log based on communication with the Bluetooth PAN. The time can be tracked for the patient wristband, the medical devices, the mobile devices, or a combination thereof. The time log can identify various interactions between the patient wristband, the medical devices, and/or the mobile devices and can be utilized for later analysis. In various embodiments, the later analysis includes analytics, patient progress, billing, reimbursement, staff scheduling, medical device usage, capacity planning, or patient acuity.

In one embodiment, the shift log component 264 of the association engine 250 is configured to communicate a shift log to the mobile device associated with the clinician. The shift log represents a time distribution for the clinician based on communication with the medical device or the signal associated with the patient via the Bluetooth PAN. The shift log component 264 may derive at least a portion of the information utilized in the shift log from the time log created by time component 262. The shift log may be utilized for later analysis including analytics, patient progress, billing, reimbursement, staff scheduling, or patient acuity.

In one embodiment, the disassociation request component 266 of the association engine 250 is configured to initiate a disassociation request for the patient from the medical device. The disassociation may be caused by an interruption in the communication of the signal or the pairing of the patient wristband 230 and one or more of the medical devices 210, 212, 214. The interruption and/or disassociation request may indicate the patient is no longer in range of the medical device, the medical device is turned off, or the patient is no longer connected to the medical device.

Once the approval component 258 receives an indication of approval from the clinician for the disassociation request, in one embodiment, the disassociation component 268 of the association engine 250 is configured to disassociate the patient from the medical device. Once disassociated, any additional communication of data from the medical device to the EMR or mobile device (other than medical device only related data, such as data not related to the patient that has been disassociated from the medical device, historical data, or subsequent association requests) is halted.

Turning now to FIG. 3, an illustrative screen display 300 is shown, in accordance with an embodiment of the present invention. Initially, a patient summary display area 310 is provided to the clinician including information corresponding to the patient (e.g., name, gender, date of birth, location). The patient summary display area may further include a categorized view of information associated with the patient. The categorized view of information may include as patient information, alerts 314, items for review 316, results 318, patient information 320, care team 322, device association 324, vitals collection 326, patient flow 328, and image capture 330. As can be appreciated, any of these categories may include data communicated by the medical devices. The categorized view of information may further be selectable to display a detailed view of the information specific to a selected category.

Referring now to FIG. 4, an illustrative screen display 400 is shown with an indication that a patient and one or more medical devices are pairing, in accordance with an embodiment of the present invention. Similar to the embodiment of FIG. 3, FIG. 4 includes a patient summary display area 410 that includes information corresponding to the patient (e.g., name, gender, date of birth, location). The identification of the patient may take place via a patient identifier included in a signal communicated to a medical device via the Bluetooth PAN, which is in turn communicated to the mobile device, in one embodiment, also via the Bluetooth PAN. A connection display area displays an indicator that the patient and one or more medical devices are pairing. As described above, pairing indicates the patient is in range of the one or more medical devices. The one or more medical devices may have detected a signal associated with the patient communicated via the Bluetooth PAN.

Turning now to FIG. 5, an illustrative screen display 500 is shown, in accordance with an embodiment of the present invention. An association confirmation display area 510 displays a prompt for a clinician to confirm an association for the one or more medical devices to the patient. The prompt may include a check box or button 512, 514, 516 for each of the one or more medical devices, allowing the clinician to selectively confirm or approve each medical device individually. Once the clinician has confirmed the association, an associate button 520 associated patients and confirmed or approved medical devices.

Referring now to FIG. 6, an illustrative screen display 600 is shown, in accordance with an embodiment of the present invention. A device display area 610 displays medical device information for the one or more medical devices. Selection of a button 612 in the device display area 610, and referring back to FIG. 5, may display medical device identifier information, location, and the like, for each of the one or more medical devices. The medical device information may assist the clinician in the confirmation or approval process or may allow the clinician to monitor data for medical devices already associated with the patient and communicated by the medical device. Selection of one of the medical devices in the device display area 522, 524, 526, such as by clicking on, hovering over, and the like may cause the device display area 522, 524, 526 to display detailed medical device information for the selected device. In one embodiment, the detailed information includes data associated with the one or more medical devices. The data may be displayed in a textual or graphical format. In one embodiment, the device display area further displays icons for the one or more medical devices enabling the clinician to distinguish between the one or more medical devices.

Turning now to FIG. 7, an illustrative screen display 700 is shown, in accordance with an embodiment of the present invention. A disconnected display area 710 displays an indicator that indicates the patient is no longer in range of the one or more medical devices, the one or more medical devices are turned off, or the one or more medical devices are disconnected from the patient. This alerts the clinician that confirmation or approval of a disassociation is needed. Selection of the disconnected display area 710, such as by clicking on, hovering over, and the like, may cause the disconnected display area 710 to reveal the disassociation confirmation display area described below.

Referring now to FIG. 8, an illustrative screen display 800 is shown, in accordance with an embodiment of the present invention. The disassociation confirmation display area 810 displays a prompt 812, 814, 816 for a clinician to confirm a disassociation for the one or more devices 822, 824, 826 from the patient. The clinician may individually confirm the disassociation and select a review button 820 to confirm or approve the disassociation.

Turning now to FIG. 9, an illustrative screen display 900 is shown, in accordance with an embodiment of the present invention. Shift log display area 910 displays a shift log associated with the clinician. The shift log represent a time distribution for the clinician based on communication with the one or more medical devices or the signal associated with the patient via the Bluetooth PAN. Shift log display area 910 may be selectable according to a particular category of clinician responsibilities, such as messaging, phone communication, patient care, and the like. Selection of a particular category may reveal details associated with that category allowing the clinician or facility to quickly assimilate and retrace the steps of the clinician. Such information can be further utilized for analytics as described herein.

Turning to FIG. 10, an illustrative flow diagram 1000 is shown of a method for automatically associating a patient to a medical device, in accordance with an embodiment of the present invention. Initially, at step 1010, a signal is received at a medical device. The signal may originate from a patient wristband associated with a patient in proximity to the medical device. The signal is communicated via a Bluetooth PAN.

At step 1020, the patient wristband associated with the signal is recognized. The patient wristband may be associated with an identifier. The identifier may be void of any patient identifying information. The patient associated with the wristband is identified at step 1030. At step 1040, the patient is associated to the medical device. In one embodiment, data associated with the medical device is communicated to an EMR associated with the patient. In one embodiment, the patient wristband and/or the medical device communicate with a mobile device associated with a clinician via the Bluetooth personal communication network.

An indication may be received indicating the patient is no longer in proximity to the medical device. In one embodiment, the indication is received when the signal is no longer being received by the medical device. In one embodiment, the indication is received when the medical device is disconnected from the patient. In one embodiment, the indication is received when the medical device is turned off. In one embodiment, the indication is received by a communication from a mobile device associated with a clinician. Once the indication is received, in one embodiment, the patient is disassociated from the medical device.

Turning to FIG. 11, an illustrative flow diagram 1100 is shown of a method for associating a medical device to a patient, in accordance with an embodiment of the present invention. Initially, a signal originating from a medical device is received by a patient wristband at step 1110. The signal is communicated via a Bluetooth PAN. At step 1120, the medical device is automatically associated to the patient. When the patient wristband no longer receives the signal, at step 1130, the medical device is automatically disassociated from the patient. In various embodiments, the patient wristband and/or the medical device communicates with a mobile device associated with a clinician via the Bluetooth PAN.

Turning to FIG. 12, an illustrative flow diagram 1200 is shown of a method for automatically associating a patient to a medical device, in accordance with an embodiment of the present invention. Initially, a signal associated with a patient is received at step 1210. The signal is communicated via a Bluetooth PAN. At step 1220, the patient associated with the signal is recognized (such as with a patient identifier associated with a patient wristband as described herein). An association request to associate the patient to a medical device is initiated at step 1230. The association request is communicated, at step 1240, to a mobile device associated with a clinician when the mobile device is in range. In one embodiment, the mobile device is automatically selected based on an assignment and proximity of the clinician. At step 1250, it is determined that the clinician has provided an indication that the association request is approved. The patient is associated to the medical device at step 1260.

Data from the medical device is automatically stored, in one embodiment, in an electronic medical record associated with the patient while the patient is associated with the medical device. In one embodiment, data is automatically logged in a shift log associated with the clinician while the mobile device associated with the clinician is communicating with the medical device or the signal associated with the patient via the Bluetooth PAN.

In one embodiment, a signal is received at the medical device indicating the patient is out of range or disconnected from the device. The signal may be that communication via the Bluetooth PAN has been interrupted. In one embodiment, this causes a disassociation request to be initiated for the patient from the medical device. The disassociation request may be communicated to the mobile device. In one embodiment, the patient is disassociated from the medical device if it is determined that the clinician has provided an indication that the disassociation request is approved.

Many different arrangements of the various components depicted, as well as components not shown, are possible without departing from the scope of the claims below. Embodiments of our technology have been described with the intent to be illustrative rather than restrictive. Alternative embodiments will become apparent to readers of this disclosure after and because of reading it. Alternative means of implementing the aforementioned can be completed without departing from the scope of the claims below. Certain features and subcombinations are of utility and may be employed without reference to other features and subcombinations and are contemplated within the scope of the claims.

The invention claimed is:

1. One or more computer storage media having computer-executable instructions embodied thereon that, when executed by one or more computing devices, cause the one or more computing devices to perform a method for automatically associating a patient to a medical device, the method comprising:
receiving a signal, at a medical device, the signal originating from a patient wristband associated with a patient in proximity to the medical device and communicated via a Bluetooth personal area network;
recognizing the patient wristband associated with the signal;
identifying a patient associated with the patient wristband; and
associating the patient to the medical device.

2. The media of claim 1, further comprising communicating data associated with the medical device to an electronic medical record associated with the patient.

3. The media of claim 1, further comprising receiving an indication the patient is no longer in proximity to the medical device.

4. The media of claim 3, wherein the indication is received when the signal is no longer being received by the medical device.

5. The media of claim 3, wherein the indication is received when the medical device is disconnected from the patient.

6. The media of claim 3, wherein the indication is received when the medical device is turned off.

7. The media of claim 3, wherein the indication is received by a communication from a mobile device associated with a clinician.

8. The media of claim 3, further comprising disassociating the patient from the medical device.

9. The media of claim 1, further comprising communicating with a mobile device associated with a clinician via the Bluetooth personal communication network.

10. A system for automatically associating a patient to a medical device, the comprising:
a patient wristband associated with a patient that communicates to one or medical devices via a Bluetooth personal area network (PAN) when the patient wristband is within range of the one or more medical devices;
the one or more medical devices configured to communicate via the Bluetooth PAN with a mobile device associated with a clinician;
the mobile device configured to receive an association request to associate the one or more medical devices to the patient; and
an electronic medical record associated with the patient configured to receive device information communicated by the one or more medical devices and clinician information from the mobile device associated with the clinician.

11. The system of claim 10, further comprising a detection component that detects a Bluetooth signal communicated from the patient wristband to the medical device or from the medical device to the patient wristband.

12. The system of claim 11, wherein communication of the Bluetooth signal causes an association request component to initiate the association request to the mobile device that includes a patient identifier associated with the patient.

13. The system of claim 12, further comprising an approval component that receives an indication from the mobile device that the clinician has approved the association request.

14. The system of claim 13, further comprising an association component that associates the patient to the medical device, enabling data from the medical device to be communicated to an electronic medical record associated with the patient.

15. The computer system of claim 10, further comprising a time component that tracks time in a time log based on communication with the Bluetooth PAN.

16. The computer system of claim 11, wherein the time log is utilized for utilized for later analysis including analytics, patient progress, billing, reimbursement, staff scheduling, medical device usage, capacity planning, or patient acuity.

17. The computer system of claim 14, wherein an interruption in the communication of the signal causes a disassociation request component to initiate a disassociation request indicating the patient is no longer in range of the medical device, the medical device is turned off, or the patient is no longer connected to the medical device.

18. The computer system of claim 17, wherein upon the approval component receiving an indication of an approval from the clinician for the disassociation request causes a disassociation component to disassociate the patient from the medical device.

19. A method for associating a medical device to a patient, the method comprising:
receiving, at a patient wristband, a signal originating from a medical device, the signal being communicated via a Bluetooth personal area network;
automatically associating the medical device to the patient; and
automatically disassociating the medical device from the patient when the patient wristband no longer receives the signal.

20. The method of claim 19, further comprising communicating with a mobile device associated with a clinician via the Bluetooth personal area network.
